(19) United States
(12) Patent Application Publication
(10) Pub. No.: US 2024/0189523 A1
(43) Pub. Date: Jun. 13, 2024

(54) HOT POROUS-SOLID METERING SYSTEMS AND METHODS FOR GENERATION OF THERAPEUTIC AEROSOLS BY EVAPORATION/CONDENSATION

(71) Applicants: The University of North Carolina at Chapel Hill, Chapel Hill, NC (US); RESEARCH TRIANGLE INSTITUTE, RESEARCH TRIANGLE PARK, NC (US)

(72) Inventors: Anthony James HICKEY, Chapel Hill, NC (US); Ian Edward STEWART, Durham, NC (US)

(21) Appl. No.: 18/286,695

(22) PCT Filed: Apr. 14, 2022
(86) PCT No.: PCT/US2022/024810

§ 371 Date: Oct. 12, 2023

Related U.S. Application Data
(60) Provisional application No. 63/174,676, filed on Apr. 14, 2021.

(51) Int. Cl. A61M 11/04

(52) U.S. Cl. CPC A61M 11/042

(57) ABSTRACT

Devices and methods for generating an aerosol of a therapeutic agent and methods of using and preparing the same are disclosed. The device includes a heatable porous substrate (e.g., a porous metal or metal alloy) embedded with a composition containing a vaporizable carrier compound (e.g., a phospholipid) and at least one therapeutic agent. The device can be incorporated into a delivery device, such as a metered dose inhaler or an exposure chamber. When the heatable porous substrate is heated, such as by resistive heating, the carrier compound is vaporized and both it and the at least one therapeutic agent are carried out of the porous substrate and, upon cooling, form aerosol particles comprising the at least one therapeutic agent.

### CROSS-REFERENCE TO RELATED APPLICATIONS

This application claims priority to U.S. Provisional Patent Application No. 63/174,676, which was filed on Apr. 14, 2021, the entire content of which is incorporated by reference herein.

### TECHNICAL FIELD

The subject matter disclosed herein relates to apparatuses and devices for generating aerosols of therapeutic agents and to methods of preparing and using the same. More particularly, the presently disclosed subject matter relates to an aerosol generation device and method utilizing a porous substrate embedded with a composition comprising a carrier compound and at least one therapeutic agent. When the porous object is heated to a vaporization temperature of the carrier compound, e.g., by using resistive heating, the carrier compound is volatilized and both the carrier compound and the at least one therapeutic agent are released from the porous object and form aerosol particles.

### BACKGROUND

Aerosol delivery is important for a number of therapeutic compounds and for treatment of certain diseases. Various techniques for generating aerosols are disclosed in U.S. Pat. Nos. 4,811,731; 4,627,432; 5,743,251; and 5,823,178, each of which is incorporated by reference herein in its entirety.

Local administration of aerosolized drugs to the airway can be useful in the treatment of pulmonary diseases, such as, but not limited to, asthma, chronic obstructive pulmonary disease (COPD), lung cancer, infectious diseases of the airway (e.g., tuberculosis and non-tuberculosis mycobacteria (NTM) infections), cystic fibrosis, pulmonary arterial hypertension, idiopathic pulmonary fibrosis, and pulmonary ciliary dyskinesia. In addition, the lung is increasingly being considered as the portal of entry for a number of aerosolized drugs designed to act systemically. The benefits of administering macromolecular aerosols have been investigated for: insulin, growth hormone, various other peptides and proteins, and gene therapeutic agents. Aerosol delivery to the airways offers advantages over other routes of administration for several disease states. Direct administration of drug to the lungs has pharmacokinetic and pharmacodynamic advantages, including greater drug concentration at the intended site of action, reduced systemic side effects, rapid and extensive drug absorption due to the large surface area of the lungs, reduced enzymatic degradation due to the lower metabolic activity of the lung, and avoidance of the first-pass metabolism effect. In addition, drug absorption and dose are not significantly affected by ingested food, patients are familiar with administration techniques, and avoidance of the disadvantages associated with injections.

However, although aerosol delivery to human subjects has been performed for over 50 years, and modified aerosol delivery systems have also been used with animal subjects, delivery systems are still surprisingly inefficient, can be difficult to use, achieve poor targeting, are irreproducible in delivery doses, and are generally inappropriate for newer applications such as gene therapy. In particular, there are often challenges and expenses, both temporal and financial, to final product development of pulmonary drug delivery systems. For example, pulmonary drug delivery systems typically employ devices that consist of a formulation, a metering system, and an aerosol generator/inhaler. The generator/inhaler is usually developed independently and adopted after optimization of the therapeutic agent. This often results in limitations of aerosol performance in terms of dose, appropriate particle size distribution for lung delivery, and stability to addressed through iterative optimization in the later stages of development.

Accordingly, there remains a long-felt, ongoing need for novel devices and methods that can produce effective aerosols of therapeutic agents for respiratory delivery to subjects.

### SUMMARY

This summary discloses several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter includes an aerosol generation apparatus or device comprising: (i) a porous heatable substrate; and (ii) a composition comprising a vaporizable carrier compound and at least one therapeutic agent; wherein the composition is embedded in pores in the porous heatable substrate; wherein when the porous heatable substrate is heated to at least the vaporization point of the vaporizable carrier, the carrier vaporizes to release the therapeutic agent from the composition and upon cooling, the carrier condenses around the at least one therapeutic agent to thereby form an aerosol comprising the at least one therapeutic agent and the carrier compound.

In some embodiments, the presently disclosed subject matter includes a metered dose inhaler comprising the aerosol generation device for use in pulmonary delivery of the at least one therapeutic agent to a subject.

In some embodiments, the presently disclosed subject matter includes a rodent nose-only exposure chamber comprising the aerosol generation device for use in pulmonary delivery of the at least one therapeutic agent to a rodent subject.

In some embodiments, the presently disclosed subject matter includes a method of producing an aerosol, comprising: (a) providing an aerosol generation device comprising: (i) a porous heatable substrate; and (ii) a composition comprising a vaporizable carrier compound and at least one therapeutic agent, wherein the composition is embedded in pores in the porous heatable substrate; (b) heating the porous heatable substrate to vaporize the vaporizable carrier compound and produce a heated vapor comprising vaporized carrier compound and the at least one therapeutic agent, thereby propelling the at least one therapeutic agent out of one or more pores in the porous heatable substrate; and (c) cooling the vapor to condense the vaporized carrier compound and the at least one therapeutic agent into an aerosol.

In some embodiments, the presently disclosed subject matter includes a method of administering a therapeutic agent to a subject, the method comprising: (a) providing an aerosol generation device comprising: (i) a porous heatable substrate; and (ii) a composition comprising a vaporizable carrier compound and at least one therapeutic agent, wherein the composition is embedded in pores in the porous heatable substrate; (b) heating the porous heatable substrate to vaporize the vaporizable carrier compound and produce a heated vapor comprising vaporized carrier compound and the at least one therapeutic agent, thereby propelling the at least one therapeutic agent out of a pore in the porous heatable substrate; and (c) cooling the vapor to condense the vaporized carrier compound and the at least one therapeutic agent into an aerosol.

Accordingly, it is an object of the subject matter disclosed herein to provide an aerosol generation device comprising a porous substrate embedded with a composition comprising a carrier compound and at least one therapeutic agent. It is also an object to provide therapeutic compounds and methods. These and other objects are achieved in whole or in part by the presently disclosed subject matter. Further, other objects and advantages of the presently disclosed subject matter will become apparent to those skilled in the art after a study of the following description, drawings and example.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the below drawings.

FIG. 1 is a schematic illustration of an example embodiment of a porous metal disc substrate.

FIG. 2 is a schematic illustration of another example embodiment of a porous metal disc comprising two regions that have different porosity from each other.

FIG. 3 is a front view of an example embodiment of an inhaler device (which can be a metered dose inhaler) for generating an aerosol from one or more porous metal discs disclosed herein.

FIG. 4 is a side view of the inhaler device shown in FIG. 3.

FIG. 5 is a rear view of the inhaler device shown in FIG. 3.

FIG. 6 is a partially exploded isometric view of the inhaler device shown in FIG. 3.

FIG. 7 is a partial internal isometric view of the inhaler device shown in FIG. 3.

FIG. 8 is an illustration of an aerosol disc assembly for use in the inhaler device shown in FIG. 3.

FIG. 9 is an illustration of the inhaler device of FIG. 3, schematically showing where the aerosol disc assembly of FIG. 8 is installed therein.

FIG. 10 is a graphical illustration showing aerodynamic particle size distribution of a Rhodamine B-containing aerosol generated.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, mass, weight, temperature, time, volume, concentration, percentage, etc., is meant to encompass variations of in some embodiments±20%, in some embodiments±10%, in some embodiments ±5%, in some embodiments±1%, in some embodiments±0.5%, and in some embodiments±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The presently disclosed subject matter relates to the delivery of therapeutic agents (e.g., pharmaceutical compounds), including small and large molecular weight drugs and biopharmaceuticals. One of the advantages provided according to the presently disclosed subject matter is an improved efficiency of the delivery of such therapeutic agents.

According to an example embodiment, a method of delivering one or more therapeutic agents is disclosed herein. This method comprises steps including exposing (e.g., directly, such as via impregnation) a substrate made from a high-heat-resistant, electrically conductive material (e.g., a metal, semiconductor, conductive polymer, etc.) to one or more biocompatible surfactant (e.g., one or more phospholipids, including those that are modified to be solid at room temperature, or about 25° C.), in which one or more therapeutic agent(s) and/or other auxiliary materials for delivery to a subject are embedded. In some embodiments, instead of or in addition to the biocompatible surfactant, other suitable vaporizable, biocompatible carrier compound that are solid at room temperative (e.g., about 25° C.) may be provided on, in, and/or about the substrate. In some embodiments, other therapeutic/delivery molecules are embedded within the biocompatible surfactant and/or the biocompatible carrier compound. The phrase “carrier compound” can be used to refer to any of the biocompatible sufactants and/or vaporizable, biocompatible carrier compounds disclosed herein, unless noted elsewhere herein.

The phase-change transition temperatures and the evaporation rates of many phospholipids are generally similar. In general, phospholipids with a longer hydrocarbon chain length have a higher melting temperature and are more hydrophobic as compared to phospholipits with comparactively shorter hycrocarbon chain lengths. The presence of lipid moieties also prevents or resists moisture ingress. In the example embodiments disclosed herein, the surfactant (e.g., one or more phospholipid) and other molecules are embedded in pores formed in the substrate, either as a mixture or sequentially (e.g., when the therapeutic agent has low solubility in the surfactant).

Passing a low voltage electrical current through the conductive material of the substrate transiently raises the temperature of the substrate temperature, which volatilizes (e.g., vaporizes) the surfactant. During, or as a result of, volatilization of the surfactant, the other embedded substances (e.g., the therapeutic agent, or agents) are propelled away from the surface of the substrate. Heating of the substrate and, therefore, also of the therapeutic agent(s), can advantageously be controlled to avoid deterioration (e.g., chemical deterioration) of the therapeutic agent(s). Due to the propelling effect of the vaporization of the surfactant, the therapeutic agent(s) do not remain in contact with the substrate long enough for degradation of the therapeutic agent(s) to occur after the vaporization temperature of the surfactant is achieved on the surface of the substrate to which the therapeutic agent(s) were attached prior to vaporization.

After the occurrence of vaporization of the surfactant, a surfactant vapor containing the therapeutic agent(s) is formed. Due to no longer being conductively heated by contact with the heated substrate, this surfactant vapor condenses (e.g., naturally, without being exposed to chilled air, such as by exposure to ambient air) and forms a lipid-coated therapeutic composition in the form of a plurality of aerosol particles. These spontaneously self-assembled aerosol particles (e.g., nano- and/or microparticles) of one or more therapeutic agents remain suspended in the air for a period of time sufficient to be inhaled by a subject (e.g., via inhalation through an inhaler) and, after such inhalation by the subject, are deposited/delivered onto a cell surface (e.g., a surface of a lung epithelial cell). The aerosol particles formed spontaneously via condensation of the surfactant vapor have a composition (e.g., defined as a ratio, or concentration, of the therapeutic agent, or agents) that is substantially proportional to the composition of the surfactant and therapeutic agent(s) embedded within the pores of the substrate.

Thus, optimization of the system can focus on aspects of aerosol formulation, aerosol delivery, and/or therapeutic targeting. Such pathways for optimization are thought to be advantageous, for from a therapeutic and regulatory perspective, as many aspects (e.g., storage stability, ability to reproduce a therapeutic outcome) are addressable simultaneously. These advantages are in direct contrast with known devices, systems, and methods for the lipid-based delivery of therapeutic agent(s), which are known to require complex studies to identify particle formulation and encapsulation efficiency/stability of the therapeutic agent(s) and any optional delivery-enhancing molecules, as well as the stability, compatibility, and deposition efficiency using a nebulizer (e.g, a vibrating-mesh nebulizer, such as a nebulizer sold under the tradename EFLOW™ (PARI Pharma GmbH, Starnberg, Germany)).

The example method disclosed herein combines aspects of composition, manufacture, and aerosol delivery of a therapeutic agent (e.g., a multi-component therapeutic agent) in a single-stage procedure, which has well-defined input parameters and can use engineering principles of heat transfer, material science, and fluid dynamics. Thus, the devices, systems, and methods disclosed herein allow for the control of aerosol particle design (e.g., composition, dose, and/or aerodynamic performance characteristics) with respect to effective therapeutic agent delivery (e.g., nucleic acid delivery) in a single process without the need for iterative empirical testing. Thus, the presently disclosed subject matter includes a novel aerosol-generating platform for the efficient and relatively economical formulation and delivery of different types of therapeutic agent(s), for example, for the treatment of lung/respiratory system diseases by providing an aerosol delivery strategy that can be used to deliver drugs to the epithelium of the airways (e.g., trachea, bronchi, lungs, etc.).

According to an example embodiment, a device (e.g., an aerosol generation and delivery platform, such as an aerosol inhaler) is provided herein. This device uses a substrate, which is made from a material comprising a plurality of pores formed therein, in which a vaporizable carrier compound (e.g., a surfactant) is contained, which acts during vaporization as a propellant for a therapeutic agent, or agents, embedded within the carrier compound to generate an aerosol for therapeutic applications, including those involving pulmonary delivery of such therapeutic agent(s). More particularly, in some embodiments, a porous, electrically conductive solid material is provided, to which a surfactant is bonded (e.g., affixed, in a solid state); at least one therapeutic agent, and, optionally, one or more additional, auxiliary substances (e.g., an absorption enhancer or cell penetration promoter) is included (e.g., in the manner of a mixture) within the carrier compound.

Preparation of aerosol particles via evaporation/condensation is described in U.S. Pat. No. 8,165,460, the disclosure of which is incorporated herein by reference in its entirety. In the heated-wire devices disclosed therein, the ability to control the dose of the therapeutic agent administered is limited to only varying the length of the wire and/or the thickness of the therapeutic agent-containing coating on the wire. According to the presently disclosed subject matter, one advantage provided is that the internal void volume (e.g., the cumulative volume of all of the pores) in the porous substrate can be used to meter (e.g., provide) a precise dosage (e.g., desired, or prescribed quantities of the therapeutic agent, or agents). As disclosed herein, substrates formed from sintered metal can be utilized for precise metering of therapeutic agent(s), but substrates are not limited to those formed only of sintered metal. In some embodiments, the substrate can be formed to have a fractal internal structure, such as can be constructed using an additive manufacturing process (e.g., binder jetting) to alter (e.g., predictably, repeatably alter) the internal voids and the dimensions of the pores formed in such an additively manufactured substrate. Thus, the presently disclosed subject matter can advantageously provide for accurate metering of doses of therapeutic agent(s), enable production-scale manufacturing, and satisfy regulatory requirements associated with the uniformity of dose delivered.

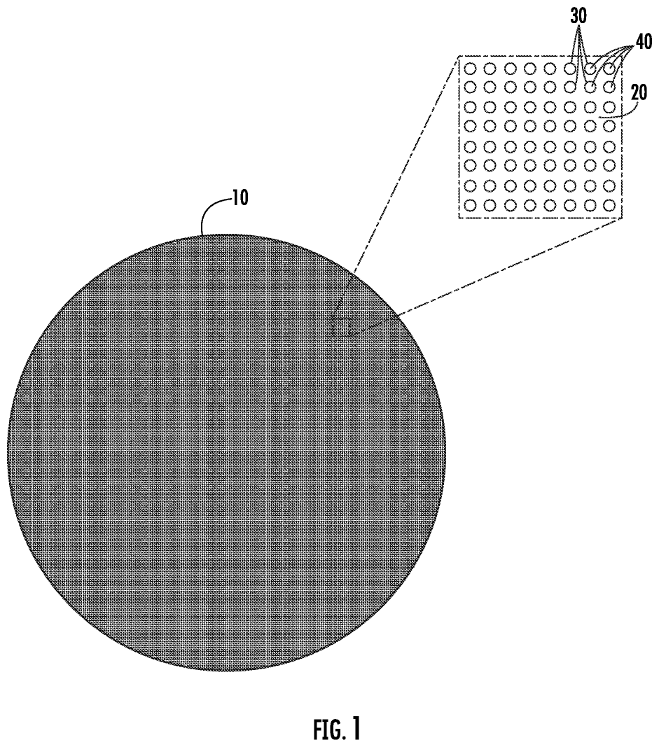

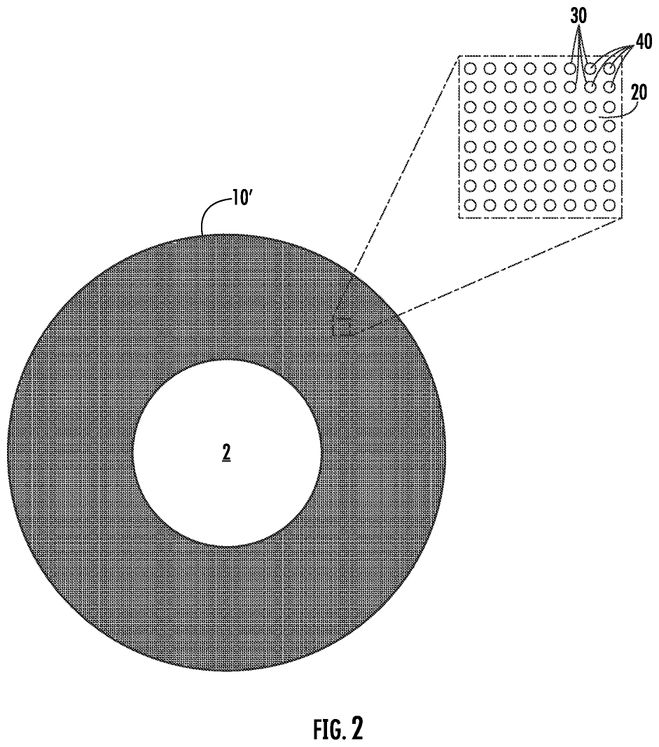

According to an example embodiment, in which the substrate comprises or consists of sintered metal(s), or metal alloy(s), sintering of powdered metals can be performed for most metals, or metal alloys, using heat and/or pressure to compact the powdered metal, thereby creating a porous, substantially solid structure in the form of a substrate. Example embodiments of a substrate formed of such sintered metal(s) or metal alloy(s) are shown in FIGS. 1 and 2, which can have any suitable shape, including geometric and/or irregular shapes.

In FIG. 1, the aerosol generation device 10 has an outer profile that is substantially entirely defined by the shape of the substrate 20, which has, as an example and without limitation to shape or size, a generally disc-like shape (e.g., having a radius that is greater than the thickness, preferably by at least a factor of 10). The substrate 20 comprises a plurality of holes, or pores 30, each of which is filled with a composition 40. As noted elsewhere herein, the composition comprises at least the vaporizable carrier compound and the therapeutic agent(s).

In FIG. 2, the aerosol generation device 10′ has an outer profile that is substantially entirely defined by the shape of the substrate 20, which has a generally annular shape (e.g., extending between an inner radius and an outer radius, which can be concentric with each other, the outer radius being greater than the thickness, preferably by at least a factor of 10). The substrate 20 comprises a plurality of holes, or pores 30, each of which is filled with a composition 40. As noted elsewhere herein, the composition comprises at least the vaporizable carrier compound and the therapeutic agent(s).

As used herein, the pores 30 can be in the form of cavities, such that the pores 30 provide the substrate 20 with an increased surface area compared to impermeable (e.g., nonporous) objects of a same size and shape. The pores 30 advantageously provide enhanced deposition of the therapeutic agent(s). Application of an electric potential (e.g., voltage) causes a flow of electric current through the substrate, which induces resistive heating of the substrate to evaporate the composition (e.g., a phospholipid, to which the therapeutic agent(s) is/are bonded) and deliver a prescribed dosage of the therapeutic agent(s) to a subject via inhalation of aerosols produced by evaporation and condensation of the carrier compound. The heating rate can be selected based on the thickness and/or resistivity of the substrate 20, in addition to controlling the voltage and current supplied to the substrate 20.

Such devices 10, 10′ can be manufactured as sintered porous metal discs (or porous metal substrates of any suitable shape) via, for example, additive manufacturing (e.g., providing enhanced internal void volume and/or geometry with custom options), heat, pressure, sponge iron process, atomization, centrifugal disintegration, liquid metal sintering, powder compaction, die pressing, isostatic compacting, shock consolidation, electric current assisted sintering, among others. The types of manufacturing processes disclosed herein are merely examples and do not limit the scope of the subject matter disclosed herein, unless stated otherwise. Such manufacturing processes can be used to create unique shapes and form factors specialized for a particular device, platform, therapeutic agent, etc. Metals, such as, but not limited to, stainless steel, nichrome, or tungsten can be used. Porous substrates can also be prepared from materials, such as, for example, semiconductor materials and/or conductive polymers.

The device 10, 10′ can be completely porous or partially porous. There can be different degrees of porosity of the device 10, 10′ (e.g., and also of the substrate 20) within the 3-dimensional volume occupied by the device 10, 10′, including substantially solid (e.g., nonporous) and hollow regions. FIG. 2 shows an example embodiment of a device 10′ comprising a substrate 20 in the form of a porous disc, where one portion 2 (e.g., the center section) of the substrate 20, is hollow, or otherwise more porous than another portion of the disc (e.g., the outer ring). The reverse can also be prepared from that which is shown in FIG. 2. In both such embodiments, the surface area and porosity of the substrate 20 can be selected based on a particular application. Porosity can be determined by gas adsorption or mercury intrusion porosimetry. The porous, semi-porous, or non-porous elements of the devices 10, 10′ can be of any desired geometry (e.g., plate, tetradedron, cube, sphere, cylinder, etc.). In some embodiments, the internal and external geometry of the devices 10, 10′ can be different from one another and/or have different degrees of porosity.

Among the advantages provided according to the presently disclosed subject matter is that, since the internal surface (e.g., surface area) defines the porosity (e.g., volume) of the device 10, 10′ that can be filled with the composition 40, a precise mass (e.g., dose of the therapeutic agent, or agents) can be controlled based on the total volume of all of the pores 30 of the substrate 20 and the density, or concentration, of the therapeutic agent(s) in the composition 40. Additionally, the electrical resistance that causes the heating of the substrate 20 is advantageously distributed throughout substantially all, or a predefined portion of, the substrate 20 to efficiently and precisely evaporate the composition from the pores 30 of the substrate 20.

Thus, the porous electrically conductive substrate can be designed with a range of surface to volume ratios as designated by well-defined porosity. This can generally follow fractal philosophy of fractal dimensions where non-integer spatial dimensions are postulated, for example, between 2 and 3 dimensions (Equation 1) to allow for enhanced therapeutic load and optimal pore filling:

D=log(N)/log(r) (Equation 1)

where D is the fractal dimension, N is the number of units, and r is the scale factor (e.g., length dimension).

The basis for fractal geometry was established on principles related to fractal dimensions between one and two dimensions (e.g., a straight line to an area). Of relevance to the practical application described herein, relationship is extrapolated to considerations of fractal dimensions between two and three dimensions (e.g., an area to a volume). The more porous a substrate 20 is, the larger the internal surface area of the substrate 20; consequently, the surface area of the substrate 20 approaches the volume of the substrate 20 at the outer bounds of increased porosity of the substrate 20.

The underlying specification of fractal geometry of self-similarity at all scales of scrutiny in producing objects with defined fractal dimensions is known traditionally in the art to be difficult, since the internal surface structure has heretofore been exceedingly difficult to control. However, using an additive manufacture process (e.g., “3D printing”), it has been found feasible to control such internal surface structure of the substrates 20. Consequently, the example embodiments described herein with regard to performance of aerosol delivery from a sintered metal disc of defined porosity demonstrates the principle of aerosol generation and delivery of therapeutic agent(s) from fractal-based solids (e.g., composition 40) bound within the pores 30 of the substrate, which is constructed to have a specific porosity corresponding to a particular application. Thus, the specific porosity, pore size, shape, quantity, and the like described herein will necessarily be altered based on a particular therapeutic application.

By varying the surface tension of the suspension, the suction pressure (e.g., capillary action) for entry into the pores 20 can used to provide for maximal loading of the composition 40 and, thus, of the therapeutic agent(s). According to Equation 2, the height of a liquid can be related to surface tension as follows:

h=(2γ)/(ρgr) (Equation 2)

where h=height of liquid, γ is the surface tension, ρ is the density of the liquid, r is the radius of curvature of the meniscus, and g is the rate of acceleration due to gravity. A high vapor pressure solvent (such as chloroform, tetrahydrofuran, acetone, methanol, hexane, pentane, diethyl ether, dichloromethane, etc.) can be included in the composition 40 to efficiently coat the substrate 20 (e.g., by substantially entirely filling the pores 30 thereof).

Any suitable therapeutic agent(s) can be delivered using the devices 10, 10′, including, small molecular weight compounds (e.g., synthetic small molecule drugs having a molecular weight of about 750 daltons or about 500 daltons or less than about 500 daltons) and/or macromolecules. such as, for example, proteins, peptides, lipids, carbohydrates, and/or nucleic acids, which can include DNA, RNA (e.g., siRNA, mRNA), and/or oligonucleotides. Additional therapeutic agent(s) can include, but are not limited to, nanoparticles, viral vectors, and/or bacteriophages.

In some embodiments, at least one therapeutic agent in the composition 40 comprises a therapeutic agent for treating or preventing pulmonary disease or disorder when administered to a subject by inhalation. In some embodiments, the pulmonary disease or disorder comprises one or more of a bacterial infection (e.g., an infection related to tuberculosis, non-tuberculosis mycobacterial infections, Legionnaires disease, whooping cough, and/or bacterial pneumonia), a viral infection (e.g., a coronavirus infection, such as a COVID-19, MERS, and/or SARS infection, an infection related to Influenza A, B, and/or C, viral pneumonia, respiratory syncytial virus, swine flu, and/or avian flu), asthma, chronic obstructive pulmonary disorder (COPD), cystic fibrosis, emphysema, bronchitis, pulmonary arterial hypertension, idiopathic pulmonary fibrosis, pulmonary ciliary dyskinesia, and/or lung cancer. The therapeutic agent(s) contained within the composition 40 comprise, but are not necessarily limited to, an anti-asthmatic, an antihistamine, an antitussive, a bronchodilator, a decongestant, an expectorant, a leukotriene modifier, a lung surfactant, an anti-infective, a corticosteroid, a mast cell stabilizer, a mucolytic, and/or a selective phosphodiesterase-4 inhibitor. In some embodiments, the therapeutic agent(s) is or comprises a nucleic acid and the devices, systems, and methods disclosed herein relate to gene therapy.

In some embodiments, one or more additional, or auxiliary non-therapeutic compounds can also be included in the composition 40 (e.g., the coating covering the substrate 20 and/or contained within, such as only within, the pores of the substrate 20) along with the one or more therapeutic agent(s). Example of such non-therapeutic compounds can include, but are not necessarily limited to, cell adhesion promoters and/or absorption enhancers. In some embodiments, the non-therapeutic compound can be provided to enhance the delivery of the therapeutic agent(s) by the subject.

The carrier compound of the composition 40 is advantageously selected, at least in part, on the vaporization properties of the carrier compound. In particular, a carrier compound is advantageously selected that has a vaporization temperature (e.g., the temperature at or above which the compound undergoes a phase change to a vapor, or gas) that is lower than the vaporization temperature of the at least one (e.g., all of the) therapeutic agent(s). In some embodiments, the vaporization temperature of the carrier compound(s) is lower than the inactivation temperature of at least one (e.g., all of the) therapeutic agent(s). For example, in some embodiments, the vaporization temperature of the carrier compound is less than 500° C., in some embodiments less than 300° ° C., and in some embodiments less than 200° C. In some embodiments, the carrier compound comprises one or more of, for example, medium chain fatty acids, polymers, amino acids, polypeptides, and/or phospholipids. Example embodiments of such carrier compounds include, but are not necessarily limited to, fatty acids (e.g., such as capric acid, lauric acid, oleic acid, palmitic acid, and stearic acid), phospholipids (e.g., including phosphatidyl cholines (PC)), polymers (e.g., including polyethylene glycols (PEG) and/or polyvinylpyrrolidone), and/or amino acids and polypeptides (e.g., including lysine, leucine, polylysine, and/or polyleucine). In some embodiments, multiple different carrier compounds can be included (e.g., blended) in the composition 40, such as to provide further control over the evaporation of the composition 40. In an example embodiment, a first carrier compound, which is liquid at room temperature and has a low vaporization temperature, is mixed with at least a second carrier compound, which has a higher vaporization temperature than the first carrier compound, to create a blended carrier compound for the composition 40, which would then have an intermediate vaporization temperature that is better suited for a particular application than either the first or second carrier compound alone.

Properties of carrier compounds suitable for use with the aerosol generation device 10, 10′ disclosed herein can include, for example and without limitation, appropriate evaporation/condensation dynamics; general acceptance in the field for use in human and animal subjects (e.g., currently marketed, FDA approved, etc.); compatibility with particular therapeutic agents, such as, for example, carrier compounds beneficial in promoting gene transfer in the airways in applications in which the therapeutic agent(s) comprises polynucleotides; no (e.g., negligible, not elevated) degradation during heating near or marginally above (e.g., 10%, 15%, or 25% above) the vaporization temperature of the carrier compound; and assisting entry of the therapeutic agent(s) into cells by, for example, transient membrane disruption, membrane fusion, and/or receptor mediated uptake (e.g., specific polypeptides).

In some embodiments, the porous (e.g., sintered) metal substrate 20 coated with the composition 40 is a disposable, or consumable (e.g., one-time-use) item. In some embodiments, the substrate 20 can be interchangeable and/or recyclable. Thus, in some embodiments, once the prescribed metered dose of the therapeutic agent(s) is delivered via inhalation to the subject, the substrate 20 can be reused (e.g., by being returned to the manufacturer and embedded with a same or different composition 40 comprising a same or different carrier compound and a same or different therapeutic agent(s)) indefinitely.

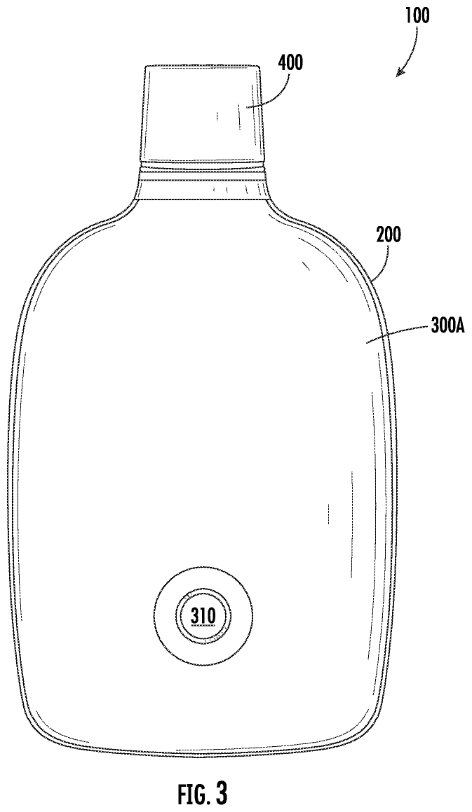

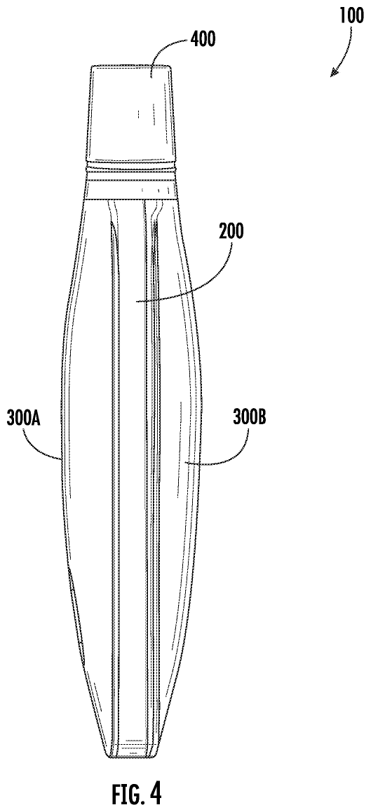

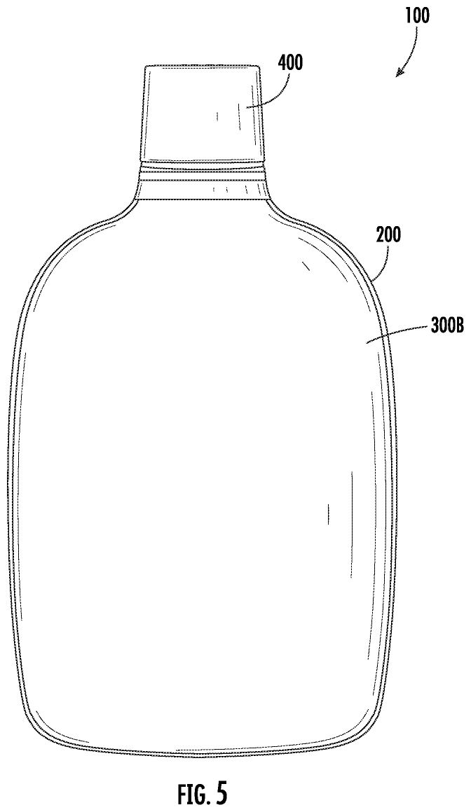

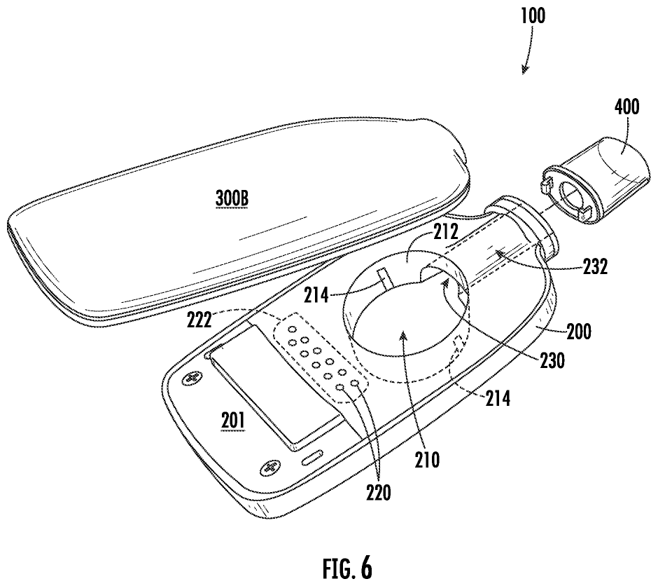

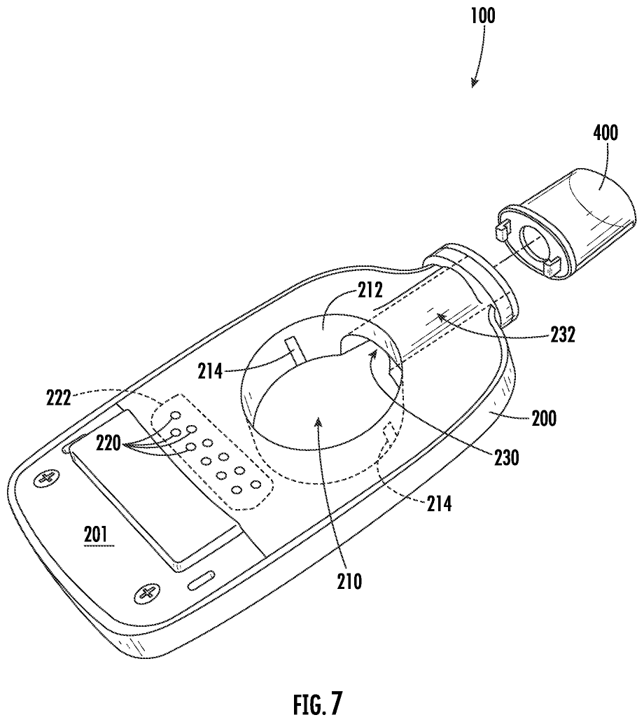

The device 10 can interface with an aerosol inhalation system via internal electronics. Aspects of an example embodiment of such an aerosol inhalation system, which for example and without limitation can be or comprise a metered dose inhaler device), generally designated 100, is shown in FIGS. 3-9. The aerosol inhalation system 100 comprises a frame 200, on opposing sides of which is provided one of the covers 300A, 300B, and a mouthpiece 400, which is connected to the frame 200. At least one of the covers (e.g., cover 300B) is removably attached to the frame 200. Formed or disposed in the frame 200 (e.g., in the thickness direction) is a cavity, generally designated 210. The cavity 210 is in fluidic communication with an inlet cavity 222 and an outlet cavity 232. The inlet cavity 222 and the outlet cavity 232 are adjacent to (e.g., directly adjacent to) the sidewall 212 of the cavity 210. The outlet cavity 232 thus forms an outlet opening, generally designated 230, in the sidewall 212 of the cavity 210.

The outlet cavity 232 extends through the frame 200 in the direction of the mouthpiece 400, which can be removably connected to the frame 200, such that the aerosol generated by the aerosol inhalation system 100 can be inhaled by a subject placing his/her mouth on, to, and/or over the mouthpiece 400 and drawing in a breath. The inlet cavity 222 is formed within the frame 200, below one or more (e.g., a plurality of) inlet holes 220 that are formed in a top or bottom surface 201 of the frame 200. The inlet cavity 222 thus extends between and provides a fluid connection (e.g., for the passage of air) between the inlet holes 220 and the cavity 210. The inlet cavity 222 is connected (e.g., directly) to the cavity 210 through and/or via the sidewall 212.

The sidewall 212 and, accordingly, the cavity 210 can have any suitable shape that allows receiving an aerosol-generating cartridge 50 within the cavity 210. In the example embodiment, the cavity 210 has a generally circular cross-sectional shape, with a volume of a disc, or truncated cylinder (e.g., having a length that is less than the radius) to receive an aerosol-generating cartridge 50 that has a substantially similar shape as the cavity 210.

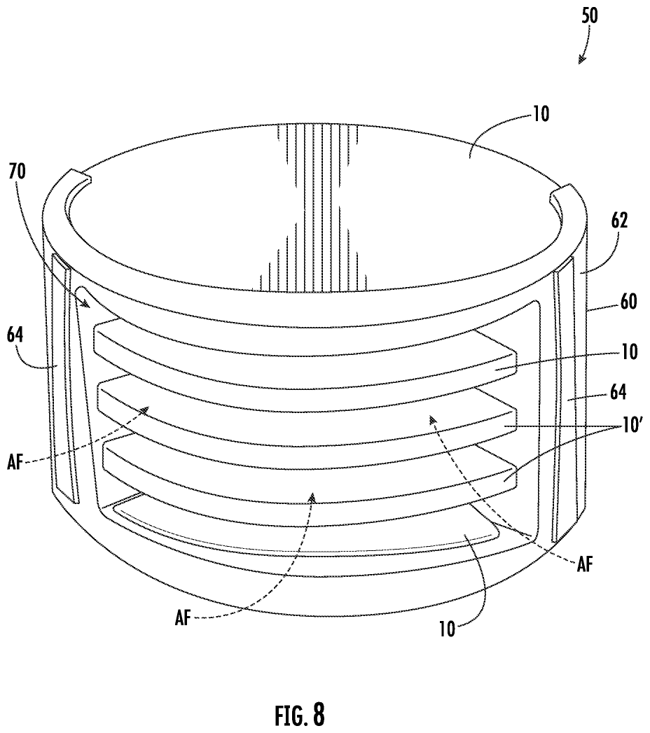

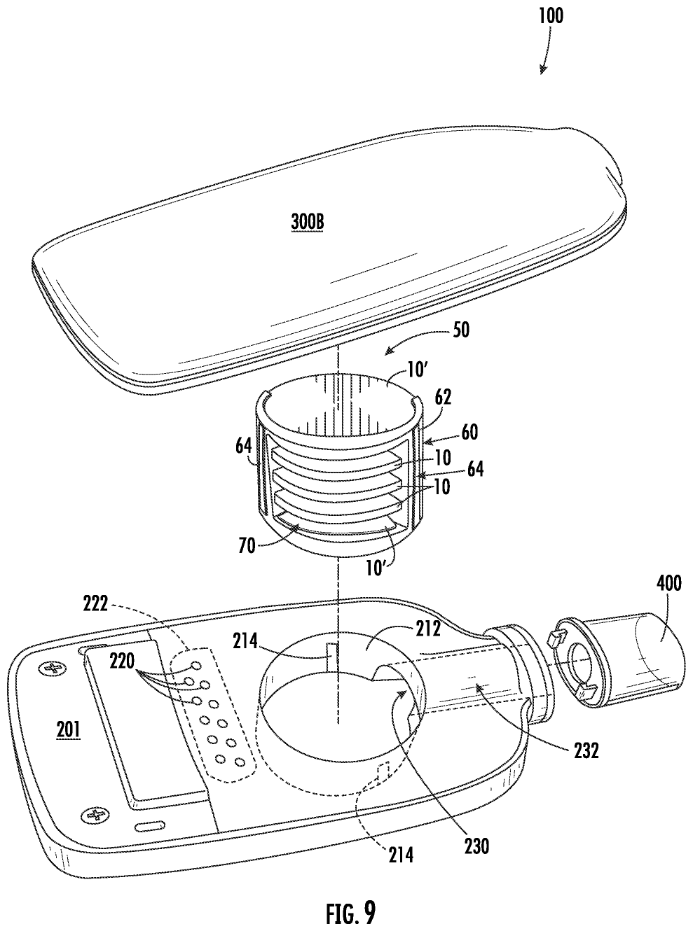

Aspects of an example embodiment of an aerosol-generating cartridge, generally designated 50, as well as the interaction of such an aerosol-generating cartridge 50 within an aerosol inhalation system 100, are shown in FIGS. 8 and 9. The aerosol-generating cartridge 50 can, for example and without limitation, comprise a cartridge frame 60, into which are inserted one or more devices 10. The cartridge frame 60 can be configured to accommodate any desired quantity of devices 10, where the cartridge 50 has a thickness that is less than the depth of the cavity 210 in the frame 200 of the aerosol inhalation system 100. The devices 10 can be removably or permanently (e.g., such as cannot be removed without damaging or deforming the cartridge frame 60) positioned within the cartridge frame 60. In the example embodiment, the cartridge frame 60 has a generally C-shaped cross-sectional profile, when viewed axially (e.g., in the direction of insertion into the cavity 210), and comprises slots configured to hold five (5) devices 5 arranged vertically on top of each other in the form of a stack, in which each device 10 is spaced apart from each vertically adjacent device 10 so as to not be in direct contact and to allow an airflow to pass through an opening, generally designated 70, and through the space AF between vertically adjacent devices 10.

The cartridge frame 60 comprises a main body 62 (e.g., the C-shaped structure) and a plurality of (e.g., two) electrodes 64. The electrodes 64 are provided in the main body 62 at positions that align, when the aerosol-generating cartridge 50 is inserted within the cavity 210, with a corresponding one of the cavity electrodes 214 provided in the sidewall 212 of the cavity 210. In FIG. 9, the aerosol-generating cartridge 50 is shown in a position in which the electrodes 64 are misaligned with the cavity electrodes 214 to better illustrate aspects of the aerosol-generating cartridge 50.

The cartridge frame 60 and the cavity 210 advantageously have keyed features that prevent insertion of an aerosol-generating cartridge 50 into the cavity 210 in which the electrodes 64 are not aligned with the cavity electrodes 214, as well as prevent rotary movement of the aerosol-generating cartridge 50 within the cavity 210 after insertion. When the aerosol-generating cartridge 50 is inserted within the cavity 210 such that the electrodes 64 are aligned with, and in contact with (e.g., direct contact with) the cavity electrodes 214, the opening 70 is aligned with the outlet opening 230. Electrical continuity between the electrodes 64 and the cavity electrodes 214 can be maintained by exerting a spring force in the direction of contact for one or both of the electrodes 64 and the cavity electrodes 214, via an interference fit between the electrodes 64 and the cavity electrodes 214, and/or via any other suitable mechanism for ensuring that electrodes 64 and the cavity electrodes 214 remain in direct contact with each other while the aerosol-generating cartridge 50 remains within the cavity 210.

The cavity electrodes 214 are electrically connected to a power source contained within the aerosol inhlation system 100, such as within the frame 200 thereof, to allow electrical current to flow from the power source, through a first electrode 64-cavity electrode 214 pair, through the device(s) 10, through a second electrode 64-cavity electrode 214 pair, and to an electrical ground. This flow of electrical current through the devices 10 induces resistive heating of the devices 10 above a vaporization temperature of the carrier compound, such that the therapeutic agent(s) from the device 10 is released into the space AF between vertically adjacent devices 10. In the example embodiment shown, the aerosol inhalation system 100 is controlled via a button 310, the pressing of which initiates the generation of aerosol, which contains the carrier compound and the therapeutic agent(s) of the composition 40, from a device 10, 10′, which are shown in FIGS. 1 and 2.

In the example embodiment shown, the aerosol-generating cartridge 50 comprises a plurality of devices 10 and a plurality of devices 10′. The devices 10, 10′ can have the same or different carrier compounds (e.g., such as would enable vaporization of the carrier compound of the devices 10 before vaporization of the carrier compound of the devices 10′), thereby allowing for a staggered administration of the therapeutic agents, which can be the same or different between the devices 10, 10′.

According to an example embodiment, a method of administering an inhaled aerosol is provided, such as by using the aerosol inhalation system 100. Such method can include inserting an aerosol-generating cartridge 50 within a cavity 210 of the frame 200 of the aerosol inhalation system 100 and installing a cover 300B to cover the aerosol-generating cartridge 50 within the cavity 210. The method can further include closing an electrical circuit to cause an electrical current to flow from the aerosol inhalation system 100 into the aerosol-generating cartridge 50 to induce a heating of the devices 10, 10′ of the aerosol-generating cartridge 50, which vaporizes the composition 40, freeing the carrier compound and the therapeutic agent(s) from the substrate 20 of the devices 10, 10′. Once no longer bound to the substrate 20, the composition 40 condenses to form an aerosol within the spaces AF between vertically adjacent devices 10, 10′. The method then includes inducing an airflow through the spaces AF, such that the aerosol is drawn out of the aerosol inhalation system 100 via a mouthpiece for administering a precise dosage of the therapeutic agent(s) to a subject using the aerosol inhalation system 100.

The aerosol inhalation system 100 can include a pressure sensor at the mouthpiece 400 of the aerosol inhalation system 100, in communication with a controller to trigger a flow of electrical current from the power supply of the aerosol inhalation system 100 to the substrate 20 of each of the devices 10, 10′ when (e.g., only when) a prescribed inspiratory flow rate is detected by the pressure sensor. The use of a pressure sensor can provide automated, or “on-demand” dosing of a subject without requiring coordination of initiation of the flow of electrical current with inhalation by the subject, such that initiation of dosing can be provided at a suitable and/ordesired moment. Furthermore, since the aerosol inhalation system 100 comprises electronics and a power source, it can be connected to wireless communication devices, such as Bluetooth® and/or WiFi® devices for data collection, such as for patient and/or clinician feedback, improved compliance/adherence to a treatment regimen, disease monitoring and control, and the like.

The subjects treated or to be treated via the devices, systems, and methods disclosed herein are desirably human subjects, although it is to be understood that the principles of the subject matter disclosed herein are suitable for use to enable effective aerosol inhalation with respect to invertebrate and to all vertebrate species, including mammals, which are intended to be included in the term “subject.” Moreover, a mammal is understood to include any mammalian species in which screening is desirable, particularly agricultural and domestic mammalian species.

The devices, systems, and methods disclosed herein are particularly useful in the treatment of warm-blooded vertebrates. Thus, the presently disclosed subject matter concerns mammals and birds.

More particularly, included herein is the treatment of mammals, such as humans, as well as those mammals of importance due to being endangered (e.g., Siberian tigers), of economical importance (e.g., animals raised on farms for consumption by humans) and/or social importance (e.g., animals kept as pets or in zoos) to humans, for instance, carnivores other than humans (e.g., cats and dogs), swine (e.g., pigs, hogs, and wild boars), ruminants (e.g., cattle, oxen, sheep, giraffes, deer, goats, bison, and camels), and horses. In some embodiments, the subject is a rodent (e.g., a mouse, rat, hamster, guinea pig, etc.). For example, inhalation exposure chambers for rodents are commonly used in toxicological studies. Also provided according to aspects of the presently disclosed subject matter is the treatment of birds, including the treatment of those kinds of birds that are endangered, kept in zoos, as well as fowl, and more particularly domesticated fowl (e.g., poultry, such as turkeys, chickens, ducks, geese, guinea fowl, and the like) as they are also of economical importance to humans. Thus, provided in the presently disclosed subject matter is the treatment of livestock, including, but not limited to, domesticated swine (pigs and hogs), ruminants, horses, poultry, and the like.

The therapeutically effective amount of a composition can depend on a number of factors. For example, the species, age, and body mass of the subject, the precise condition requiring treatment and its severity, the nature of the composition, and the route of administration of the composition are all factors that can be considered.

The devices, systems, and methods disclosed herein can also be useful as adjunctive, add-on, or supplementary therapy for a disease, such as one of the pulmonary diseases/disorders disclosed elsewhere herein. The adjunctive, add-on, or supplementary therapy means the concomitant or sequential administration of therapeutic agent(s), according to the devices, systems, and methods disclosed herein, to a subject who has already received administration of, who is receiving administration of, and/or who will receive administration of one or more additional, or “second” therapeutic agents or treatments (e.g., surgery, radiation, and/or an orally, subcutaneously, or intravenously administered therapeutic compound).

### EXAMPLE

An example embodiment of a device 10 is described hereinbelow to further illustrate various aspects of the presently disclosed subject matter. However, those of ordinary skill in the art should, in light of the present disclosure, appreciate that many changes can be made in, to, and/or regarding the example embodiments disclosed herein and still obtain a like or similar result without departing from the spirit and scope of the presently disclosed subject matter.

The device 10 comprises a substrate 20 made from a porous 316L stainless steel sintered disc having the following dimensions: 1 cm diameter, 1.5 mm thickness, and 2 μm pore size. This substrate 20 was used in producing the following experimental results. Other metals, dimensions, and pore sizes can be used. A fluorescent dye (rhodamine B) was used as the example therapeutic agent and a phospholipid (lecithin) was used as the example carrier compound. Thus, the composition 40 comprised the fluorescent dye and the phospholipid. The dye and phospholipid were dissolved in chloroform at 0.1 mg/mL and 1 mg/mL, respectively, resulting in a dye:lipid mass ratio of 10:1. Two successive 0.1 mL aliquots of this solution were deposited on the stainless steel disc (one on each side). The disc was impregnated, and the solvent was evaporated in less than 5 seconds for both applications of the solution onto the substrate. The coatings of the solution onto the substrate were performed in a chemical hood. Electrical connections (e.g., “alligator”-style clips) were made on the device 10 and the device 10 was set in place above a viable 6-stage Andersen Cascade Impactor (ACI) using a custom polycarbonate “inlet.” Vacuum was applied to the ACI (28 L/min+/−2 L/min) for five seconds, after which the DC power supply (XFR 20-60 Xantrex) connected to the device 10 was switched on to apply 2 V, which resulted in a current flow of 40 A. Noticeable aerosol was viewable after ˜5 seconds and the power supply remained on for another 30 seconds while more aerosol was qualitatively produced. Power supply and vacuum were switched off after about 35 seconds and the device 10 was carefully disconnected from the power source.

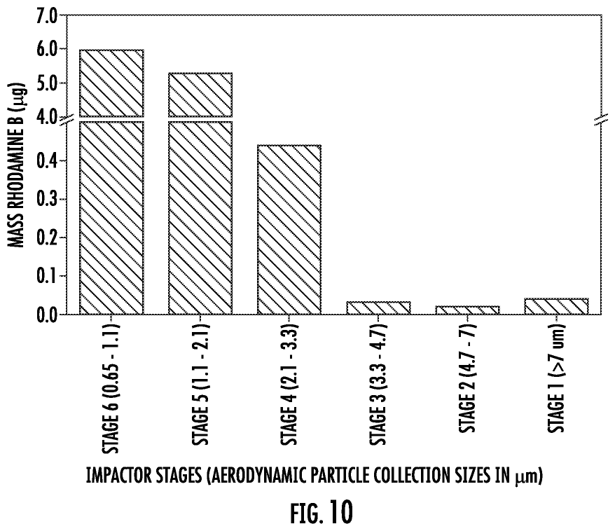

The plates of the ACI were assayed for rhodamine B content via fluorescence spectroscopy (excitation=545 nm; emission=575 nm). Stages were washed with EtOH. FIG. 10 shows the resulting data indicating that >99% of the collected rhodamine existed as particles with aerodynamic diameter less than 3.3 μm—within the aboaut 1 μm to about 10 μm size range suitable for aerosol delivery in humans. The graph shown in FIG. 10 shows the mass of Rhodamine B (in micrograms (μg)) versus aerodynamic particle size (in micrometers (μm)) in particles collected in different stages of the Andersen Cascade Impactor (ACI) after aerosol generation.

In some embodiments, release of the composition 40 from the substrate 20 can be achieved using electromechanical excitation. The vaporization temperature for many phospholipids is within the range of about 38-42° C. Consequently, a movement and/or vibration can be used, in some embodiments, either instead of, or along with, the flow of electrical current for vaporization of the composition 40 from the surface of the substrate 20. Using movement and/or vibration, the temperature of the substrate is increased sufficiently to vaporize the carrier compound and, after condensation, produce aerosol droplets. One example of using such movement and/or vibration of the substrate 20 for vaporization of the carrier compound includes use of a piezoelectric device that transmits vibration to the substrate 10 to both increase the temperature of the substrate 10 above the vaporization temperature of the carrier compound and, simultaneously, disperse the vaporized carrier compound droplets, after increasing in temperature above their vaporization temperature, into air, where such vaporized carrier compound droplets would solidify at room temperature to form an aerosol suitable for therapeutic inhalation by a subject. The use of movement and/or vibration instead of the electrothermal heating discussed elsewhere herein is advantageous, at least in some instances, as it allows for more precise control of the temperature of the substrate to avoid the composition 40 being exposed to temperatures significantly (e.g., 10%, 15%, 20%, 25%) above the vaporization temperature during heating of the substrate by, for example, a piezoelectric device. Thus, via the use of movement and/or vibration, the composition 40 can advantageously be exposed to lower temperatures than using electrothermal heating, which would allow for administration as aerosol of therapeutic agent(s) comprising less temperature-stable molecules.

It will be understood that various details of the presently disclosed subject matter may be changed without departing from the scope of the presently disclosed subject matter. Furthermore, the foregoing description is for the purpose of illustration only, and not for the purpose of limitation.

1. A device for aerosol generation comprising:
a substrate comprising a porous material having a plurality of pores distributed throughout the substrate; and
a composition, which comprises a carrier compound and at least one therapeutic agent, wherein the composition is embedded in the plurality of pores of the substrate;
wherein the device is configured such that, when the substrate is heated at or above a vaporization temperature of the carrier compound, the carrier compound is vaporized;
wherein, when the carrier compound is vaporized, the at least one therapeutic agent is released from the composition; and
wherein, as the carrier compound cools, the carrier compound is configured to condense around the at least one therapeutic agent, by which an aerosol is formed, the aerosol comprising the at least one therapeutic agent and the carrier compound.

2. The device of claim 1, wherein the porous material of the substrate comprises a sintered metal and/or a sintered metal alloy, optionally, wherein the sintered metal and/or sintered metal alloy comprises stainless steel, a nickel-chrome alloy, tungsten, aluminum, and/or titanium.

3. The device of claim 1, wherein the substrate comprises a first region, which has a first porosity, and a second region, which has a second porosity, the second porosity being different from the first porosity.

4. The device of claim 1, wherein the substrate is formed via an additive manufacturing process.

5. The device of claim 1, wherein the plurality of pores of the substrate comprises a total volume of internal void space, which is a same size or greater than a volume of the composition, the volume of the composition comprising a therapeutic dosage of the at least one therapeutic agent for administration to a subject.

6. The device of claim 1, wherein the substrate comprises a disc or a cylinder.

7. The device of claim 1, comprising a power source electrically connected to the substrate and configured to apply an electrical current to the substrate.

8. The device of claim 1, wherein the vaporization temperature of the carrier compound is less than a vaporization temperature of each of the at least one therapeutic agent.

9. The device of claim 8, wherein the vaporization temperature of the carrier compound is less than about 500° C.

10. The device of claim 1, wherein the carrier compound comprises a phospholipid or lecithin.

11. The device of claim 1, wherein the at least one therapeutic agent comprises a small molecule, a polynucleotide, a polypeptide, a nanoparticle, a viral vector, and/or a non-viral vector.

12. The device of claim 1, wherein the at least one therapeutic agent comprises a therapeutic agent for treatment of a pulmonary disease or disorder, optionally, wherein the pulmonary disease or disorder comprises a bacterial infection, a viral infection, asthma, chronic obstructive pulmonary disorder (COPD), cystic fibrosis, emphysema, bronchitis, pulmonary arterial hypertension, idiopathic pulmonary fibrosis, primary ciliary dyskinesia, and/or lung cancer.

13. The device of claim 1, wherein the composition embedded in the plurality of pores of the substrate comprises one or more additives, optionally, the one or more additives comprising an absorption enhancer.

14. A metered dose inhaler comprising at least one device according to claim 1, wherein the metered dose inhaler is configured for pulmonary delivery of the at least one therapeutic agent to a subject.

15. A rodent nose-only exposure chamber comprising at least one device according to claim 1, wherein the exposure chamber is configured for pulmonary delivery of the at least one therapeutic agent to a rodent subject.

16. The exposure chamber of claim 15, comprising an elutriator positioned in fluid communication between the at least one device and the exposure chamber.

17. A method of producing an aerosol, the method comprising:
providing a device for generating the aerosol comprising:
a substrate comprising a porous material having a plurality of pores distributed throughout the substrate; and
a composition, which comprises a carrier compound and at least one therapeutic agent, wherein the composition is embedded in the plurality of pores of the substrate;
heating the substrate to a vaporization temperature of the carrier compound to produce a vaporized carrier compound and the at least one therapeutic agent, wherein formation of the vaporized carrier compound propels the at least one therapeutic agent out of the plurality of pores in the substrate; and
cooling, after vaporization, the vaporized carrier compound, which causes the vaporized carrier compound and the at least one therapeutic agent to condense, thereby forming an aerosol comprising the at least one therapeutic agent and the carrier compound.

18. The method of claim 17, wherein the porous material of the substrate comprises a sintered metal and/or a sintered metal alloy, optionally, wherein the sintered metal and/or sintered metal alloy comprises stainless steel, a nickel-chrome alloy, tungsten, aluminum, and/or titanium.

19. The method of claim 17, wherein the substrate comprises a first region, which has a first porosity, and a second region, which has a second porosity, the second porosity being different from the first porosity.

20. The method of claim 17, wherein providing the device comprises forming the substrate via an additive manufacturing process.

21. The method of claim 17, wherein the plurality of pores of the substrate comprises a total volume of internal void space, which is a same size or greater than a volume of the composition, the volume of the composition comprising a therapeutic dosage of the at least one therapeutic agent for administration to a subject.

22. The method of claim 17, wherein providing the device comprises:
contacting the substrate with a volume of a solution and/or suspension, the solution and/or suspension comprising the carrier compound and the at least one therapeutic agent, wherein the solution and/or suspension comprises a high vapor pressure solvent, and wherein the solution and/or suspension comprises a surface tension, by which the solution and/or suspension flows into the plurality of pores of the substrate, optionally via capillary action; and
evaporating the high vapor pressure solvent, such that only the composition remains embedded in the plurality of pores of the substrate.

23. The method of claim 17, wherein the carrier compound comprises a phospholipid or lecithin.

24. The method of claim 17, wherein the at least one therapeutic agent comprises a therapeutic agent for treating a pulmonary disease or disorder.

25. The method of claim 17, wherein heating the substrate comprises a resistive heating of the substrate via a flow of electric current through the substrate.

26. The method of claim 17, wherein cooling the vaporized carrier compound comprises exposing the vaporized carrier compound to ambient air.

27. A method of administering at least one therapeutic agent to a subject, the method comprising:
providing a device for generating an aerosol comprising:
a substrate comprising a porous material having a plurality of pores distributed throughout the substrate; and
a composition, which comprises a carrier compound and the at least one therapeutic agent, wherein the composition is embedded in the plurality of pores of the substrate;
heating the substrate to a vaporization temperature of the carrier compound to produce a vaporized carrier compound and the at least one therapeutic agent, wherein formation of the vaporized carrier compound propels the at least one therapeutic agent out of the plurality of pores in the substrate;
cooling, after vaporization, the vaporized carrier compound, which causes the vaporized carrier compound and the at least one therapeutic agent to condense, thereby forming an aerosol comprising the at least one therapeutic agent and the carrier compound; and
providing the aerosol to the subject via inhalation of the aerosol by the subject.

28. The method of claim 27, wherein the device is provided in a metered dose inhaler.
